(19) United States
(12) Patent Application Publication
(10) Pub. No.: US 2007/0167382 A1
(43) Pub. Date: Jul. 19, 2007

(54) Crystalline and amorphous forms of telithromycin

(76) Inventors: Nina Finkelstein, Herzliya (IL); Ben-Zion Dolitzky, Petach Tiqva (IL); Judith Aronhime, Rehovot (IL); Sigalit Levi, Modi'in (IL)

Correspondence Address: KENYON & KENYON LLP, ONE BROADWAY, NEW YORK, NY, 10004, US

(21) Appl. No.: 11/600,675

(22) Filed: Nov. 15, 2006

Related U.S. Application Data
(60) Provisional application No. 60/737,091, filed on Nov. 15, 2005.
(60) Provisional application No. 60/740,398, filed on Nov. 28, 2005.
(60) Provisional application No. 60/752,433, filed on Dec. 20, 2005.

(51) Int. Cl. A61K 31/7052; C07H 17/08

(52) U.S. Cl. 514/29; 536/7.4

(57) ABSTRACT

Provided is telithromycin which melts at a range of 175° C. to 185° C. Also provided are solid states of telithromycin and processes for the preparation thereof.

### CROSS-REFERENCE TO RELATED APPLICATIONS

This application claims the benefit of priority to U.S. provisional application Ser. Nos. 60/737,091,filed Nov. 15, 2005; 60/740,398,filed Nov. 28, 2005;and 60/752,433,filed Dec. 20, 2005,hereby incorporated by reference.

### FIELD OF THE INVENTION

The invention encompasses telithromycin which melts at a range of 175° C. to 185° C. The invention also encompasses solid states of telithromycin and processes for the preparation thereof.

### BACKGROUND OF THE INVENTION

Telithromycin is a ketolide antimicrobial agent. KETEK™ tablets contain telithromycin, a semisynthetic antibacterial in the ketolide class for oral administration. Chemically, telithromycin is designated as Erythromycin, 3-de[(2,6-dideoxy-3-C-methyl-3-O-methyl-(alpha)-L-ribo-hexopyranosyl)oxy]-11,12-dideoxy-6-O-methyl-3-oxo-12,11-[oxycarbonyl[[4-[4-(3-pyridinyl)-1H-imidazol-1-yl]butyl]imino]]. Telithromycin, a ketolide, differs chemically from the macrolide group of antibacterials by the lack of (alpha)-L-cladinose at position 3 of the erythronolide A ring, resulting in a 3-keto function. It is further characterized by a C₁₁₋₁₂ carbamate substituted by an imidazolyl and pyridyl ring through a butyl chain. Its empirical formula is C₄₃H₆₅N₅O₁₀ and its molecular weight is 812.03.Telithromycin is a white to off-white crystalline powder. The following represents the chemical structure of telithromycin.

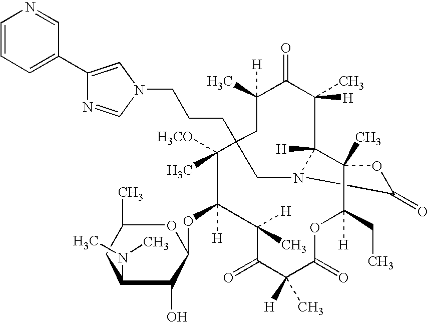

U.S. Pat. No. 5,635,485 and corresponding European Patent No. EP 596,802 disclose methods for the preparation of crude telithromycin. As disclosed therein, the obtained crude product was then purified by chromatography on silica, eluting with a CH₂Cl₂—MeOH—NH₄OH mixture (95-5-0.4). Crystallization from diethyl ether reportedly gave the product having a melting point of 187° C.-188° C.

International Publication No. WO 2005/105821 (“WO '821”) also discloses methods for the preparation of telithromycin. In Example 19 of WO '821, telithromycin was reportedly obtained by recrystallization from a mixture of methyl-tert-butyl ether and cyclohexanone. WO '821,p. 26, 11. 14-26.

Polymorphism, the occurrence of different crystal forms, is a property of some molecules and molecular complexes. A single molecule, like telithromycin, may give rise to a variety of crystalline forms having distinct crystal structures and physical properties like melting point, x-ray diffraction pattern, infrared absorption fingerprint, and solid state NMR spectrum. One crystalline form may give rise to thermal behavior different from that of another crystalline form. Thermal behavior can be measured in the laboratory by such techniques as capillary melting point, thermogravimetric analysis (“TGA”), and differential scanning calorimetry (“DSC”), which have been used to distinguish polymorphic forms.

The difference in the physical properties of different crystalline forms results from the orientation and intermolecular interactions of adjacent molecules or complexes in the bulk solid. Accordingly, polymorphs are distinct solids sharing the same molecular formula yet having distinct advantageous physical properties compared to other crystalline forms of the same compound or complex.

One of the most important physical properties of pharmaceutical compounds is their solubility in aqueous solution, particularly their solubility in the gastric juices of a patient. For example, where absorption through the gastrointestinal tract is slow, it is often desirable for a drug that is unstable to conditions in the patient's stomach or intestine to dissolve slowly so that it does not accumulate in a deleterious environment. Different crystalline forms or polymorphs of the same pharmaceutical compounds can and reportedly do have different aqueous solubilities.

As mentioned, polymorphs differ in physical characteristics influenced by the conformation and orientation of the molecules in the unit cell. These physical characteristics can be, for example, thermal behavior, stability, and hygroscopic properties.

The discovery of new polymorphic forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic. There is a need in the art for polymorphic forms of telithromycin.

### SUMMARY OF THE INVENTION

One embodiment of the present invention encompasses telithromycin which melts at a range of 175° C. to 185° C. Preferably, the telithromycin is in crystalline form.

Another embodiment of the invention encompasses anhydrous telithromycin which melts at a range of 175° C. to 185° C. Preferably, the anhydrous telithromycin is in crystalline form.

Another embodiment of the invention encompasses an amorphous form of telithromycin.

Another embodiment of the invention encompasses crystalline telithromycin characterized by X-ray powder diffraction peaks at 12.0, 12.7, 15.8, 17.0 and 19.6 degrees two-theta±0.2 degrees two-theta.

Another embodiment of the invention encompasses pure crystalline telithromycin, characterized by an X-ray diffraction pattern free of a peak at about 7.7° degrees two-theta±0.2 degrees two-theta.

Another embodiment of the invention encompasses crystalline telithromycin, characterized by X-ray powder diffraction peaks at 11.9, 12.1, 15.8, 18.0 and 23.8 degrees two-theta±0.2 degrees two-theta.

Yet another embodiment of the invention encompasses processes for preparing amorphous and crystalline forms of telithromycin.

### BRIEF DESCRIPTION OF THE FIGURES

FIG. 1 illustrates a powder X-ray diffraction pattern for crystalline telithromycin Form A.

FIG. 2 illustrates a powder X-ray diffraction pattern for crystalline telithromycin Form B.

FIG. 3 illustrates a powder X-ray diffraction pattern for amorphous telithromycin.

FIG. 4 illustrates a differential scanning calorimetry thermogram of crystalline telithromycin Form A.

FIG. 5 illustrates a differential scanning calorimetry thermogram of crystalline telithromycin Form B.

FIG. 6 illustrates a differential scanning calorimetry thermogram of amorphous telithromycin.

FIG. 7 illustrates a differential scanning calorimetry thermogram of pure crystalline telithromycin Form A.

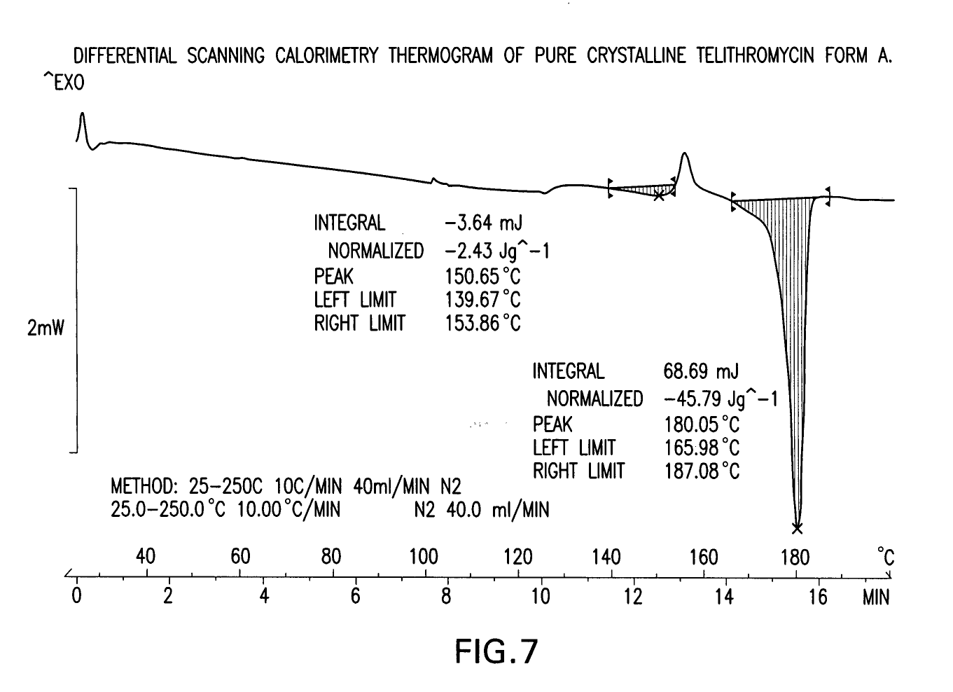

FIG. 8 illustrates a powder X-ray diffraction pattern for pure crystalline telithromycin Form A, crystalline telithromycin Form B and crystalline telithromycin Form A. (The powder X-ray diffraction peaks of Form B found in the Form A diffractogram are marked by arrows.)

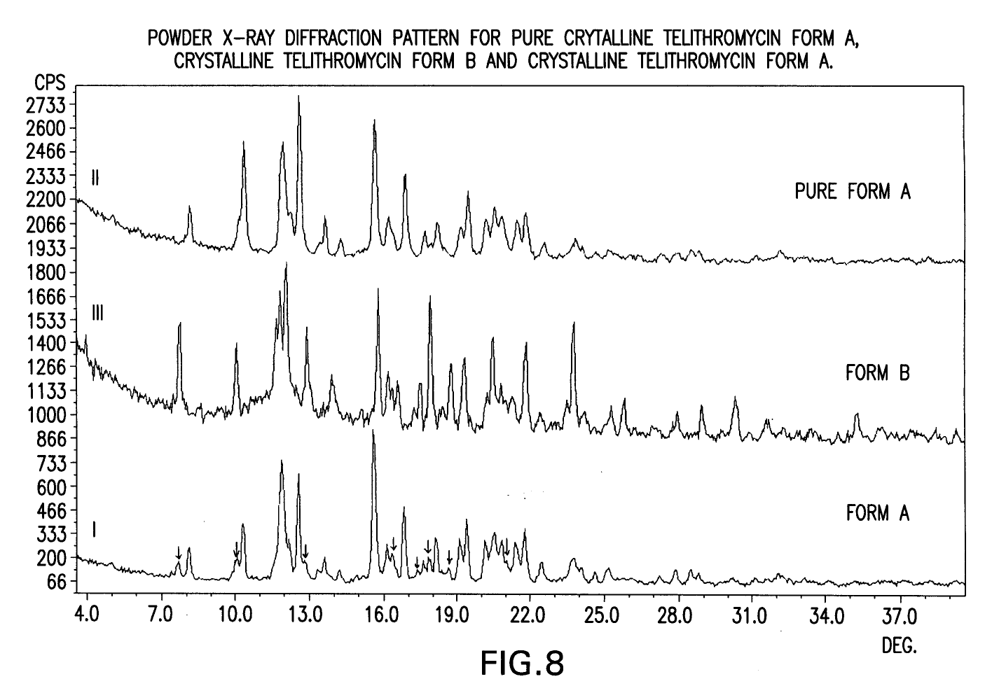

### DETAILED DESCRIPTION OF THE INVENTION

Amorphous solids consist of disordered arrangements of molecules and do not possess a distiguishable crystal lattice. An amorphous solid generally is more soluble than its crystalline form, leading to a more rapid bioavailability. Lack of peaks in a powder XRPD pattern or lack of an endothermic melting peak in a DSC thermogram may indicate presence of an amorphous form. The area under the peaks in an XRPD pattern may be added to obtain total amount of crystalline material. In a DSC thermogram the presence of endotherms may point to the melting of crystalline material.

As used herein, unless otherwise defined, the term “anhydrous” refers to telithromycin containing 1% by weight or less of water or other solvent. That is, anhydrous telithromycin of the invention has a total solvent content of less than 1% by weight.

As used herein, unless otherwise defined, the term “gel” refers to a semi solid chemical mixture which resembles jelly.

As used herein, unless otherwise defined, the term “room temperature” refers to a temperature of about 20° C. to about 25° C.

The invention encompasses telithromycin which melts at a range of 175° C. to 185° C. Preferably, the telithromycin is in crystalline form. The telithromycin which melts at a range of 175° C. to 185° C. of the invention has the advantage of stability in humid conditions, as demonstrated in tables 1 and 2.

The invention also encompasses anhydrous telithromycin having a melting point at a range of 175° C. to 185° C. Preferably, the anhydrous telithromycin is in crystalline form.

The invention also encompasses amorphous telithromycin. Preferably, the amorphous telithromycin has less than about 20% by weight of crystalline telithromycin, more preferably less than about 10% by weight of crystalline telithromycin, and most preferably less than about 1% by weight of crystalline telithromycin.

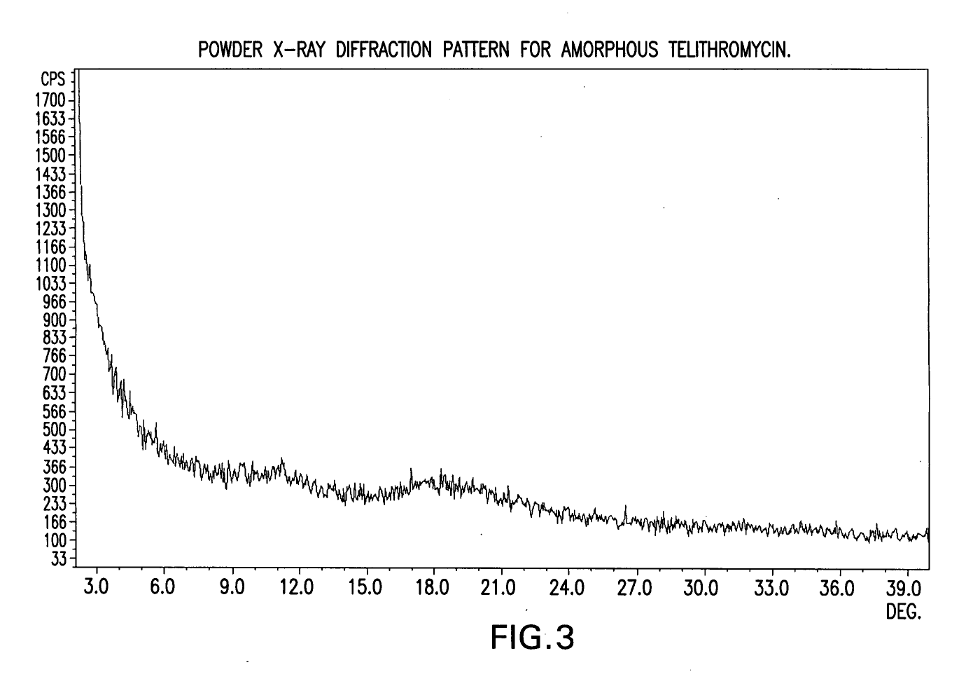

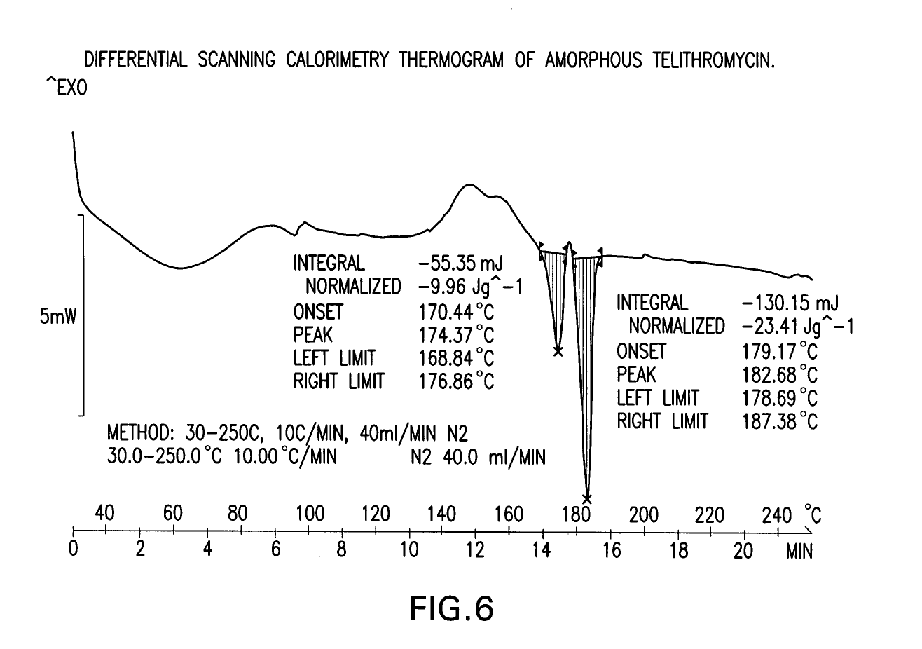

The amorphous telithromycin has an X-ray diffraction pattern typical for an amorphous solid. The X-ray diffraction of amorphous telithromycin shows a halo-pattern lacking visible crystalline peaks, as illustrated in FIG. 3. Further, FIG. 6 shows a representative thermogram from differential scanning calorimetry (“DSC”) for amorphous telithromycin with peaks at about 174° and about 183° C.

The invention also encompasses a process for preparing amorphous telithromycin. The amorphous telithromycin may be prepared by precipitation from water. In one embodiment, telithromycin and water are combined to form a mixture containing amorphous telithromycin; and the amorphous telithromycin is recovered from the mixture. Typically, the mixture of telithromycin and water is heated at a temperature of about 70° C. to reflux to obtain a solution, and the solution is cooled to obtain a precipitate of amorphous telithromycin, which is then recovered. Typically, the mixture is maintained at room temperature for a period of about 1 hour to about 5 days to obtain the amorphous telithromycin

The amorphous telithromycin may be recovered by any method known to one of skill in the art. Such methods include, but are not limited to, filtering the solution to isolate the precipitated amorphous telithromycin, followed by drying the isolated amorphous telithromycin.

The invention also encompasses a process for preparing amorphous telithromycin comprising: heating a mixture of telithromycin and methyl tert butyl ether (“MTBE”) to form a solution; cooling the solution to room temperature to obtain a gel; evaporating the ether to obtain amorphous telithromycin; and recovering the amorphous telithromycin. Preferably, the mixture of telithromycin and the ether is heated to reflux to form the solution.

The amorphous telithromycin may be recovered by any method known to one of skill in the art. Such methods include, but are not limited to, drying the precipitate. Preferably, the precipitate is dried at about 50° C. for about 16 hours at a pressure below about 100 mmHg in a vacuum oven to obtain amorphous telithromycin.

The invention also encompasses a process for preparing amorphous telithromycin comprising: exposing telithromycin to a solvent, wherein the solvent is a C₂₋₆ alcohol, to obtain amorphous telithromycin; and recovering the amorphous telithromycin.

Preferably, the solvent is isopropanol.

Typically, the telithromycin is exposed to the solvent for a period of time sufficient to form amorphous telithromycin. Preferably, the telithromycin is exposed to the solvent for about 1 day to about 40 days, more preferably for about 40 days. Preferably, the telithromycin is exposed to the solvent at a temperature of about 15° C. to about 35° C.

Amorphous telithromycin may be recovered by any method known to one of skill in the art. Such methods include, but are not limited to, drying. Preferably, the obtained amorphous telithromycin is dried at a temperature of about 50° C. for about 16 hours at a pressure below about 100 mmHg in a vacuum oven.

Amorphous telithromycin of the invention has the advantage of stability upon contacting with various solvents, as demonstrated in Examples 23-31. As described below, amorphous telithromycin retains its physical structure, even after suspension in various solvents or precipitation from various solvents.

For example, providing a suspension of amorphous telithromycin and a solvent selected from the group consisting of water and heptane; and maintaining the suspension for about 48 hours at room temperature results in recovery of amorphous telithromycin.

Providing a suspension of amorphous telithromycin and a solvent system of methanol/water, acetonitrile/water, ethanol/water, acetone/water, 2-propanol/water or tetrahydrofuran/water at room temperature in closed tubes; maintaining the suspension for about 4 days to obtain an emulsion; and maintaining the obtained emulsion in open tubes for another 4 days results in obtaining a gel from which amorphous telithromycin is recovered. Preferably, the ratio of solvents used to form the suspension is about 1:1 (volume:volume).

Furthermore, providing a solution of amorphous telithromycin in dioxane, followed by precipitation from hexane, results in recovery of amorphous telithromycin.

Amorphous telithromycin may be recovered by any method known to one of skill in the art. Such methods include, but are not limited to, drying the gel to recover amorphous telithromycin. Preferably, the gel is dried at about 50° C. for about 16 hours at a pressure below about 100 mmHg in a vacuum oven to obtain amorphous telithromycin.

The invention further encompasses crystalline forms of telithromycin, which may be characterized by at least one of weight loss measured by thermogravimetric analysis (“TGA”) or by X-Ray powder diffraction (“XRPD”). Preferably, the crystalline forms of telithromycin described herein contain not more than 20% (w/w) of other crystalline forms of telithromycin and preferably not more than 10%.

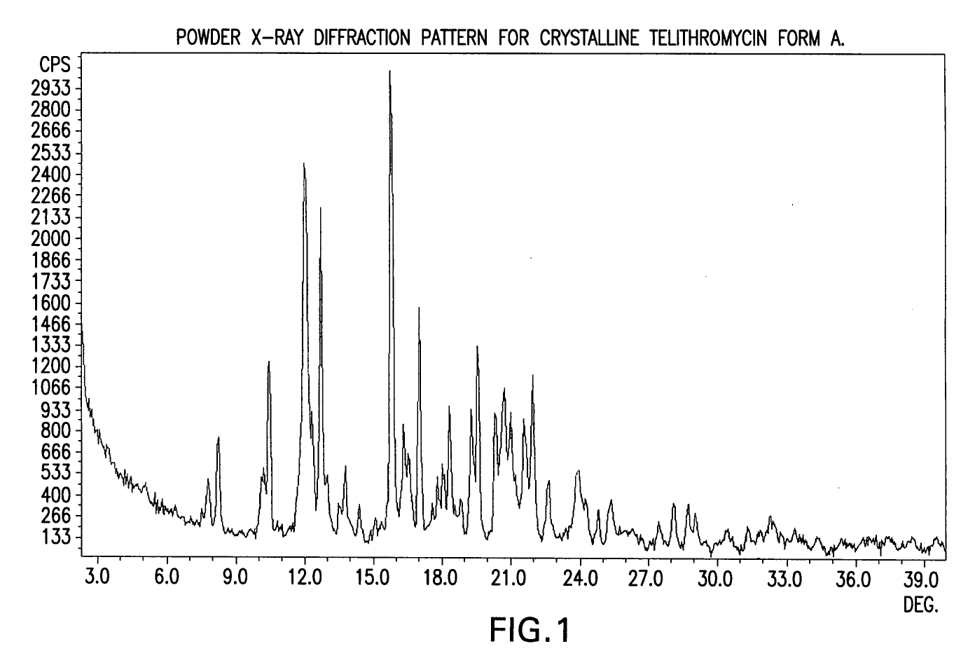

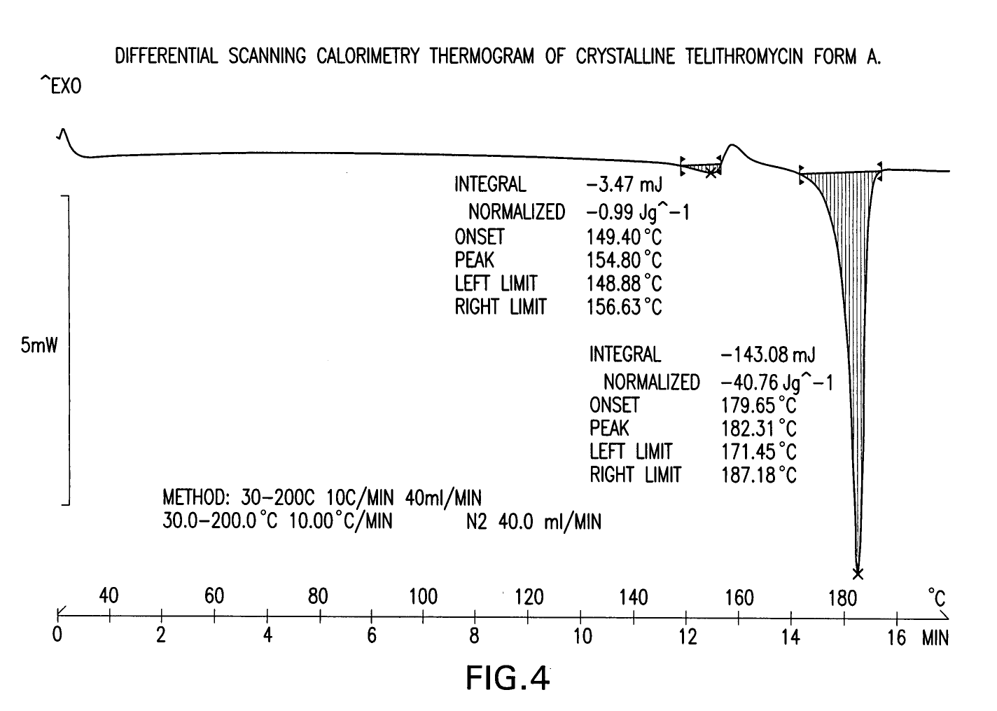

The invention encompasses crystalline telithromycin, herein defined as Form A, characterized by an X-ray powder diffraction pattern having peaks at 12.0°, 12.7°, 15.8°, 17.0°, and 19.6° 2θ±0.2° 2θ. Form A may be further characterized by an X-ray powder diffraction pattern having peaks at 8.2°, 10.4°, 18.3°, 20.7°, and 21.9° 2θ±0.2° 2θ, substantially as depicted in FIG. 1. Form A may also be identified by a differential scanning calorimetry thermogram with peaks at about 155° and about 182° C., substantially as depicted in FIG. 4.

Preferably, crystalline telithromycin Form A is anhydrous.

Crystalline telithromycin Form A may be prepared by a process comprising: providing a suspension of amorphous telithromycin in hexane to obtain crystalline telithromycin Form A; and recovering the crystalline telithromycin Form A.

Typically, the suspension is maintained for a period of time sufficient to obtain crystalline telithromycin Form A. Preferably, the suspension is maintained for about 15 to about 48 hours to obtain crystalline telithromycin Form A. Preferably, the suspension is maintained at room temperature.

Crystalline telithromycin Form A may also be prepared by precipitation from a mixture of amorphous telithromycin and hexane, with or without water. In one embodiment, amorphous telithromycin and hexane, with or without water, are heated at a temperature of about 50° C. to about 120° C. to obtain a mixture, followed by removal of the solvent to obtain crystalline telithromycin Form A and recovering the crystalline telithromycin Form A. Typically, the mixture is maintained for at least about half an hour before removing the solvent. Typically, the solvent is removed by evaporation while heating at a temperature of about 30° C. to about 80° C.

The crystalline telithromycin Form A may be recovered by any method known to one of skill in the art. Such methods include, but are not limited to, filtering and drying the precipitate. Preferably, the precipitate is dried at a temperature of about 40° C. to about 80° C. for at least about 5 hours, more preferably for about 16 to about 24 hours, at a pressure below about 100 mmHg in a vacuum oven to obtain crystalline telithromycin Form A.

Crystalline telithromycin Form A may also be prepared by crystallization from a solvent/anti-solvent system. This process comprises providing a solution of telithromycin in a cyclic, branched, or unbranched C₄-C₁₀ ether; combining the solution with heptane to obtain a precipitate of crystalline telithromycin Form A; and recovering the precipitated crystalline telithromycin Form A.

Typically, the telithromycin starting material is amorphous telithromycin.

Preferably, the ether is 2-methyl tetrahydrofuran.

The crystalline telithromycin Form A may be recovered by any method known to one of skill in the art. Such methods include, but are not limited to, filtering and drying the precipitate. Preferably, the precipitate is dried at a temperature of from about 50° C. for about 16 hours, at a pressure below about 100 mmHg in a vacuum oven to obtain crystalline telithromycin Form A.

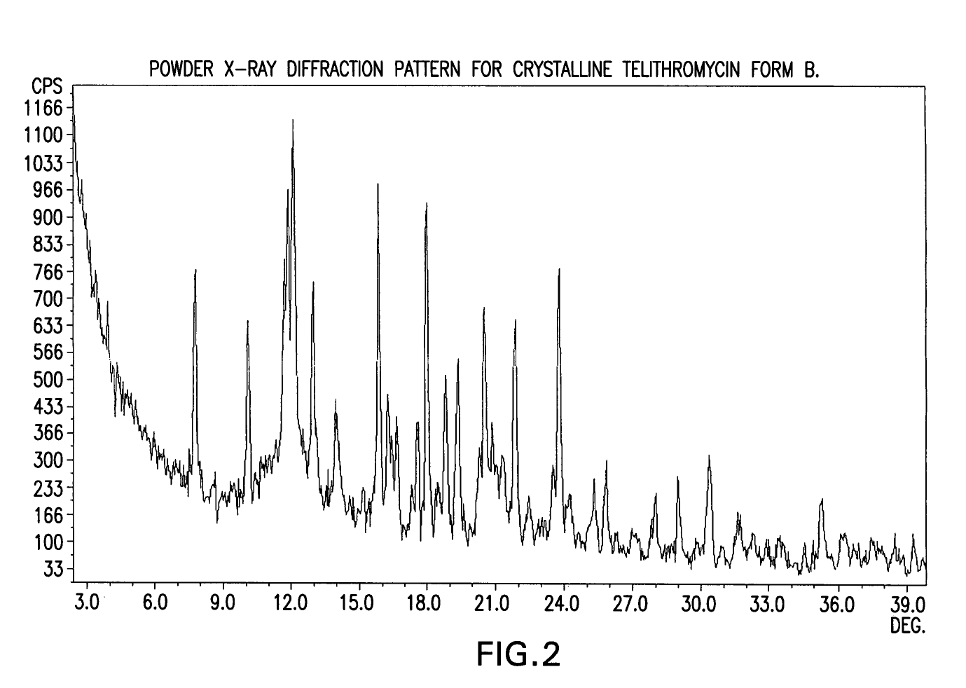

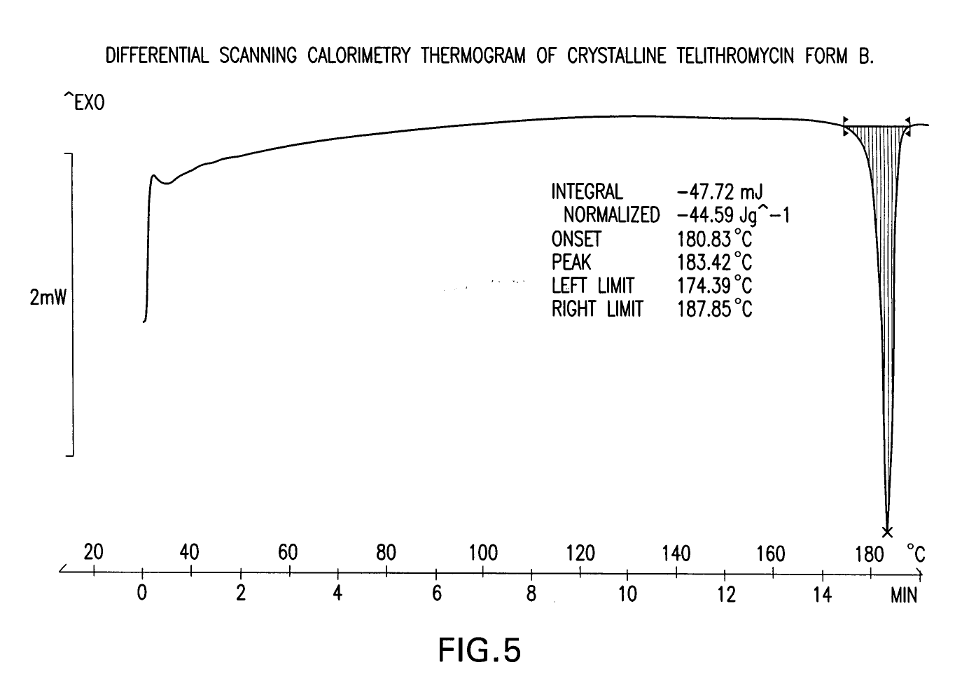

The invention also encompasses crystalline telithromycin, herein defined as Form B, characterized by an X-ray powder diffraction pattern having peaks at 11.9°, 12.1°, 15.8°, 18.0°,and 23.8° 2θ±0.2° 2θ. Form B may be further characterized by an X-ray powder diffraction pattern having peaks at 7.8°, 10.1°, 12.9°, 16.4°, 17.6°, 20.5°, 21.3°,and 21.9° 2θ±0.2° 2θ, substantially as depicted in FIG. 2. Form B may also be identified by a differential scanning calorimetry thermogram with a peak at about 183° C., substantially as depicted in FIG. 5.

Preferably, crystalline telithromycin Form B is anhydrous.

Crystalline telithromycin Form B may be prepared by a process comprising: providing a suspension of amorphous telithromycin in a solvent, wherein the solvent is heptane or an aliphatic, branched, or unbranched C₄-C₁₀ ether; and recovering the crystalline telithromycin Form B from the suspension.

Preferably, the solvent is heptane, diisopropylether, or diethyl ether. Typically, the amorphous telithromycin and solvent are heated to form the suspension. Preferably, the amorphous telithromycin and solvent are heated at a temperature of about 40° C. to about reflux temperature of the solvent to form the suspension.

Typically, the suspension is maintained for a period of time sufficient to obtain crystalline telithromycin Form B. Preferably, the suspension is maintained for at least about 1 hour to obtain crystalline telithromycin Form B. More preferably, the suspension is maintained for about 1 hour to about 5 hours to obtain crystalline telithromycin Form B.

The crystalline telithromycin Form B may be recovered by any method known to one of skill in the art. Such methods include, but are not limited to, filtering and drying the telithromycin Form B. Preferably, the crystalline telithromycin Form B is dried at a temperature of from about 40° C. to about 80° C. for at least about 5 hours, more preferably for about 16 to about 24 hours, at a pressure below about 100 mmHg in a vacuum oven.

Crystalline telithromycin Form B may also be prepared by a process comprising: providing a suspension of amorphous telithromycin in a solvent system of heptane/water, heptane/isopropanol or hexane/isopropanol; and recovering crystalline telithromycin Form B from the suspension.

Typically, the ratio of solvents in the solvent system is between about 40:1 (volume:volume) and about 500:1 (volume:volume).

Typically, the amorphous telithromycin and the solvent system are heated to form the suspension. Preferably, the amorphous telithromycin and the solvent system are heated at a temperature of about 40° C. to about 120° C. to form the suspension

Typically, the suspension is maintained for a period of time sufficient to obtain crystalline telithromycin Form B. Preferably, the suspension is maintained for a period of at least about 4.5 hours to obtain crystalline telithromycin Form B.

Crystalline telithromycin Form B may also be prepared by precipitation from a mixture of amorphous telithromycin and aliphatic, branched, or unbranched C₄-C₁₀ ether, which is not MTBE. In one embodiment, the amorphous telithromycin and the ether are heated at a temperature of about 40° C. to about 120° C. to obtain a mixture, followed by removal of the ether to obtain Form B and recovery of Form B. Typically, the mixture is maintained for at least 4.5 hours before removing the ether. Generally, the ether is evaporated while heating at a temperature of from about 30° C. to about 80° C.

The crystalline telithromycin Form B may be recovered by any method known to one of skill in the art. Such methods include, but are not limited to, drying the crystalline telithromycin Form B. Preferably, the crystalline telithromycin Form B is dried at about 50° C. for about 16 hours at a pressure below about 100 mmHg in a vacuum oven.

Crystalline telithromycin Form B may also be prepared by crystallization from a solvent/anti-solvent system. This process comprises providing a solution of telithromycin in a solvent, wherein the solvent is a cyclic branched or unbranched C₄-C₁₀ ether or C₆-C₈ aromatic hydrocarbon; combining the solution with hexane to obtain crystalline telithromycin Form B; and recovering the crystalline telithromycin Form B.

Typically, the telithromycin starting material is amorphous telithromycin.

Preferably, the solvent is 2-methyl tetrahydrofuran or toluene.

Crystalline telithromycin Form B may also be prepared by crystallization from an aliphatic, branched, or unbranched C₄-C₁₀ ether. This process comprises providing a solution of telithromycin in the ether and precipitating crystalline telithromycin Form B from the solution. Typically, the telithromycin and ether are heated to facilitate dissolution of the telithromycin. Preferably, the telithromycin and ether are heated at the reflux temperature of the solvent to obtain the solution. Typically, the precipitation of crystalline telithromycin Form B is induced by cooling the solution to a temperature of about 0° C. Preferably, the solution is maintained at a temperature of about 0° C. for about 5 hours to obtain a precipitate.

Typically, the telithromycin starting material is amorphous telithromycin.

Preferably, the solvent is diethyl ether.

The invention also encompasses a process for preparing crystalline telithromycin Form B comprising: exposing telithromycin to a solvent, wherein the solvent is a C₄₋₁₀ ether, to obtain crystalline telithromycin Form B; and recovering the crystalline telithromycin Form B.

Preferably, the solvent is diethyl ether or di-isopropyl ether.

Typically, the telithromycin is exposed to the solvent for a period of time sufficient to form crystalline telithromycin Form B. Preferably, the telithromycin is exposed to the solvent for about 1 day to about 40 days and more preferably for about 40 days. Preferably, the telithromycin is exposed to the solvent at a temperature of about 15° C. to about 35° C.

Crystalline telithromycin Form B may be recovered by any method known to one of skill in the art. Such methods include, but are not limited to, drying. Preferably, the obtained form is dried at a temperature of about 50° C. for about 16 hours at a pressure below about 100 mmHg in a vacuum oven.

Crystalline telithromycin Form B may also be prepared by a process comprising heating crystalline telithromycin Form A or amorphous telithromycin. Preferably, crystalline telithromycin Form A or the amorphous telithromycin is heated at a temperature of about 25° C. and about 160° C. to obtain crystalline telithromycin Form B. More preferably, crystalline telithromycin Form A is heated in a differential scanning calorimetry furnace with an initial temperature of 30° C. while increasing the temperature at a rate of about 10° C. per minute until a final temperature of 160° C. is reached. Alternatively, amorphous telithromycin is heated in a differential scanning calorimetry furnace with an initial temperature of 25° C. while increasing the temperature at a rate of about 10° C. per minute until a final temperature of 160° C. is reached.

The invention also encompasses pure crystalline telithromycin Form A having less than 10% by weight of crystalline telithromycin Form B. Pure crystalline telithromycin Form A is characterized by an X-ray powder diffraction pattern free of a detectable peak at about 7.7° 2θ±0.2° 2θ. See U.S. PHARMACOPEIA, 2402 (27th ed. 2004). The peak at about 7.7° 2θ is a characteristic peak of crystalline telithromycin Form B.

Pure crystalline telithromycin Form A may be prepared by a process comprising: exposing amorphous telithromycin to a solvent, wherein the solvent is a C₅₋₈ aliphatic or aromatic hydrocarbon or a mixture of a C₅₋₈ aliphatic or aromatic hydrocarbon and water, to obtain pure crystalline telithromycin Form A; and recovering pure crystalline telithromycin Form A.

Preferably, the solvent is hexane, pentane, or a mixture thereof with water. More preferably, the solvent is hexane. When a mixture of a C₅₋₈ aliphatic or aromatic hydrocarbon and water is used, the hydrocarbon /water ratio is preferably about 98:2 to about 99:1.

Typically, the amorphous telithromycin is exposed to the solvent for a period of time sufficient to form pure crystalline telithromycin Form A. Preferably, the amorphous telithromycin is exposed to the solvent for about 1 day to about 40 days, more preferably for about 40 days. Preferably, the amorphous telithromycin is exposed to the solvent at a temperature of about 15° C. to about 35° C.

Pure crystalline telithromycin Form A may be recovered by any method known to one of skill in the art. Such methods include, but are not limited to, drying. Preferably, the obtained form is dried at a temperature of about 60° C. for about 7 hours at a pressure below about 100 mmHg in a vacuum oven.

Crystalline telithromycin Forms A and B and amorphous telithromycin were exposed to 80% relative humidity (“RH”) for 24 hours at room temperature (“RT”). The exposed samples were analyzed by XRPD. The results are summarized in Tables 1-3:

TABLE 1
Stability results of telithromycin crystalline Form A
Hygroscopicity at 80% RH for 24 hours at RT

| Sample: | Crystal form (by XRPD) |
|---|---|
| Initial form (before the exposure): | Form A |
| Final form (after the exposure): | Form A |

TABLE 2
Stability results of telithromycin crystalline Form B
Hygroscopicity at 80% RH for 24 hours at RT

| Sample: | Crystal form (by XRPD) |
|---|---|
| Initial form (before the exposure): | Form B |
| Final form (after the exposure): | Form B |

TABLE 3
Stability results of amorphous telithromycin
Hygroscopicity at 80% RH for 24 hours at RT

| Sample: | Crystal form (by XRPD) |
|---|---|
| Initial form (before the exposure): | Amorphous |
| Final form (after the exposure): | Amorphous |

The results in Tables 1-3 demonstrate that crystalline telithromycin Form A and Form B and amorphous telithromycin are all stable at 80% humidity for 24 hours at room temperature.

Having described the invention with reference to certain preferred embodiments, other embodiments will become apparent to one skilled in the art from consideration of the specification. The invention is further defined by reference to the following examples describing in detail the analysis of the telithromycin crystalline forms and methods for preparing the crystalline forms of the invention. It will be apparent to those skilled in the art that many modifications, both to materials and methods, may be practiced without departing from the scope of the invention.

### EXAMPLES

X-Ray powder diffraction data were obtained by using method known in the art using a SCINTAG powder X-Ray diffractometer model X'TRA equipped with a solid-state detector. Copper radiation of 1.5418 Å was used. A round aluminum sample holder with zero background was used. The scanning parameters included: range: 2° to 40° 2θ;scan mode: continuous scan; step size: 0.05°;and a rate of 3°/min.

All peak positions are within ±0.2° 2θ.

Differential scanning calorimetry (“DSC”) analysis was done using a Mettler 821 Stare. The weight of the samples is about 2 mg. The samples were scanned at a rate of 10° C./min from 30° C. to 200° C. The oven is constantly purged with nitrogen gas at a flow rate of 40 ml/min. Standard 40 ml aluminum crucibles covered by lids with three holes were used.

Melting points are determined by the peak temperature of the DSC curve.

### Example 1

### Preparation of Pure Crystalline Telithromycin Form A

Amorphous telithromycin (10 mg) was put in a glass tube. The tube was put into a bigger closed vessel (the vessel volume 125 ml), containing 20 ml of hexane. After 40 days a sample from the solid was analyzed by XRPD and found to be pure crystalline telithromycin Form A.

Then the sample was dried in a vacuum oven at 60° C. for 7 hours. The dry sample was analyzed by XRPD and found to be pure crystalline telithromycin Form A. The melting point measured by DSC is 180° C.

### Example 2

### Preparation of Crystalline Telithromycin Form A

Amorphous telithromycin (20 mg) was suspended in hexane (0.2 ml) and kept at ambient temperature over the week end. A sample from the suspension was analyzed by XRPD and found to be Form A. The melting point measured by DSC is 184° C.

### Example 3

### Preparation of Crystalline Telithromycin Form A

Amorphous telithromycin (20 mg) was heated at 70° C. in hexane (0.2 ml) in a closed high pressure tube. After 1 hour the hexane was evaporated during the heating. After additional 3.5 hours the solid was cooled to ambient temperature. A sample from the solid was analyzed by XRPD and found to be crystalline telithromycin Form A.

The sample was then dried in a vacuum oven at 50° C. for 16 hours. The dry sample was analyzed by XRPD and found to be crystalline telithromycin Form A.

### Example 4

### Preparation of Crystalline Telithromycin Form A

Amorphous telithromycin (20 mg) was heated at 70° C. in hexane (0.2 ml), containing 0.2 volume % of water in a closed high pressure tube. After ½ an hour the solvent was evaporated during the heating. After additional 4 hours the solid was cooled to ambient temperature. A sample from the solid was analyzed by XRPD and found to be crystalline telithromycin Form A. The melting point measured by DSC is 182° C.

### Example 5

### Preparation of Crystalline Telithromycin Form A

Amorphous telithromycin (100 mg) was dissolved in 2-methyl tetrahydrofuran (0.3 ml). Heptane (1.5 ml) was added to the solution, and telithromycin was precipitated. A sample from the solid was analyzed by XRPD and found to be crystalline telithromycin Form A.

The sample then was dried in a vacuum oven at 50° C. for 16 hours. The dry sample was analyzed by XRPD and found to be telithromycin Form A.

### Example 6

### Preparation of Crystalline Telithromycin Form B

Amorphous telithromycin (20 mg) was heated at 70° C. in heptane (0.2 ml) in a closed high pressure tube for 4.5 hours. The suspension was cooled to ambient temperature. A sample from the suspension was analyzed by XRPD and found to be crystalline telithromycin Form B.

### Example 7

### Preparation of Crystalline Telithromycin Form B

Amorphous telithromycin (20 mg) was heated at 70° C. in diethyl ether (0.2 ml) in a closed high pressure tube for 4.5 hours. Diethyl ether was evaporated during the heating. The solid was cooled to ambient temperature. A sample from the solid was analyzed by XRPD analysis and found to be crystalline telithromycin Form B.

The sample was then dried in a vacuum oven at 50° C. for 16 hours. The dry sample was analyzed by XRPD and found to be crystalline telithromycin Form B. The melting point measured by DSC is 183° C.

### Examples 8-10

### Preparation of Crystalline Telithromycin Form B

Amorphous telithromycin (20 mg) was heated at 70° C. in solvent (0.2 ml) in a closed high pressure tube for 4.5 hours. The mixture was cooled to ambient temperature. A sample from the suspension was analyzed by XRPD and found to be crystalline telithromycin Form B. The weight loss of example 8 measured by TGA is 0.87%.

| Example | Solvents (volume:volume) |
|---|---|
| 8 | Heptane/water (500:1) |
| 9 | Heptane/isopropanol (100:1) |
| 10 | Hexane/isopropanol (100:1) |

### Example 11

### Preparation of Crystalline Telithromycin Form B

Amorphous telithromycin (0.5 g) was heated at reflux with stirring in diethyl ether (130 ml) for an hour. The suspension was cooled to ambient temperature and the solid was filtered. The wet solid was analyzed by XRPD and found to be crystalline telithromycin Form B.

The solid was then dried in a vacuum oven at 50° C. for 16 hours, analyzed by XRPD and found to be crystalline telithromycin Form B.

### Example 12

### Preparation of Crystalline Telithromycin Form B

Amorphous telithromycin (20 mg) was suspended in diisopropylether (16ml) and heated to reflux. The suspension was cooled to ambient temperature. A sample from the suspension was analyzed by XRPD and found to be crystalline telithromycin Form B.

The sample was then dried in a vacuum oven at 65° C. for a weekend. The dry sample was analyzed by XRPD and found to be crystalline telithromycin Form B.

### Example 13

### Preparation of Crystalline Telithromycin Form B

Crystalline telithromycin Form A (2 mg) was heated in the DSC (Mettler 821 Star^{e}) at the range of 30°-160° C. at a rate of 10°/min. The DSC furnace is constantly purged with nitrogen gas at a flow rate of 40 ml/min. The heated sample was analyzed by XRPD and found to be crystalline telithromycin Form B.

### Example 14

### Preparation of Telithromycin Form B

Amorphous telithromycin (1.8 mg) was heated in the DSC (Mettler 821 Star^{e}) at the range of 25°-160° C. at a rate of 10°/min. The DSC furnace is constantly purged with nitrogen gas at a flow rate of 40 ml/min. The heated sample was analyzed by XRPD and found to be crystalline telithromycin Form B.

### Example 15

### Preparation of Telithromycin Form B

Amorphous telithromycin (50 mg) was dissolved in 2-tethyltetrahydrofuran (0.5 ml). Hexane (1.5 ml) was added to the solution, and telithromycin precipitated. The wet solid was analyzed by XRPD and found to be crystalline telithromycin form B.

### Example 16

### Preparation of Telithromycin Form B

Amorphous telithromycin (20 mg) was dissolved in toluene (0.2 ml). Hexane (0.2 ml) was added to the solution, and telithromycin precipitated. The wet solid was analyzed by XRPD and found to be crystalline telithromycin form B

### Example 17

### Preparation of Telithromycin Form B

Amorphous telithromycin (35 mg) was dissolved in diethyl ether by heating at reflux. The solution was then cooled to 0° C. and was left to stand at 0° C. for 5 hours, during which time a solid precipitated from the solution. A sample from the precipitated solid was analyzed by XRPD and was found to be crystalline telithromycin Form B.

The sample then was dried in a vacuum oven at 50° C. for 16 hours. The dry sample was analyzed by XRPD and found to be crystalline telithromycin Form B.

### Example 18

### Preparation of Telithromycin Form B

Telithromycin (10 mg) was put in a glass tube. The tube was put into a bigger closed vessel (the vessel volume 125 ml), containing 20 ml of diethyl ether. After 40 days a sample from the solid was analyzed by XRPD and found to be crystalline telithromycin Form B.

Then the sample was dried in a vacuum oven at 50° C. for 16 hours. The dry sample was analyzed by XRPD and found to be crystalline telithromycin Form B.

### Example 19

### Preparation of Telithromycin Form B

Telithromycin (10 mg) was put in a glass tube. The tube was put into a bigger closed vessel (the vessel volume 125 ml), containing 20 ml of di-isopropyl ether. After 40 days a sample from the solid was analyzed by XRPD and found to be crystalline telithromycin Form B.

Then the sample was dried in a vacuum oven at 50° C. for 16 hours. The dry sample was analyzed by XRPD and found to be crystalline telithromycin Form B.

### Example 20

### Preparation of Amorphous Telithromycin

Telithromycin (20 mg) was heated at 70° C. in water (0.2 ml) in a closed high pressure tube for an hour for dissolution. After additional 3.5 hours the solution was cooled to ambient temperature, and was left to stand overnight. A sample from the suspension was analyzed by XRPD and found to be amorphous telithromycin.

### Example 21

### Preparation of Amorphous Telithromycin

Telithromycin (0.5 g) was heated at reflux with stirring in MTBE (12 ml). The solution was cooled to ambient temperature and gave a gel. The solvent was evaporated. A wet sample was analyzed by XRPD and found to be amorphous telithromycin.

Then the sample was dried in a vacuum oven at 50° C. for 16 hours. A dry sample was analyzed by XRPD analysis and found to be amorphous telithromycin.

### Example 22

### Preparation of Amorphous Telithromycin

Telithromycin (10 mg) was put in a glass tube. The tube was put into a bigger closed vessel (the vessel volume 125 ml), containing 20 ml of iso-propanol. After 40 days a sample from the solid was analyzed by XRPD and found to be amorphous telithromycin.

Then the sample was dried in a vacuum oven at 50° C. for 16 hours. The dry sample was analyzed by XRPD and found to be amorphous telithromycin.

### Example 23

### Stability of Amorphous Telithromycin in Dioxane and Hexane

Amorphous telithromycin (50 mg) was dissolved in dioxane (0.05 ml). Hexane (1.5 ml) was then added to the solution, and telithromycin precipitated. The wet solid was analyzed by XRPD and found to be amorphous telithromycin.

### Example 24

### Stability of Amorphous Telithromycin in Heptane

Amorphous telithromycin (20 mg) was suspended in heptane (0.2 ml) and kept at ambient temperature over the weekend. A sample from the suspension was analyzed by XRPD and found to be amorphous.

### Example 25

### Stability of Amorphous Telithromycin in Water

Amorphous telithromycin (20 mg) was suspended in water (0.2 ml) and kept at ambient temperature over the weekend. A sample from the suspension was analyzed by XRPD and found to be amorphous telithromycin. No melting peak was detected by DSC.

### Examples 26-31

### Stability of Amorphous Telithromycin in a Mixture of Solvents

Amorphous telithromycin (20 mg) was suspended in solvent (0.2 ml) and kept in closed tubes at room temperature for 4 days. Then the obtained emulsion was kept in the opened tube for 4 days and gave a gel. The gel was dried in a vacuum oven at 50° C. for 16 hours. A dry sample was analyzed by XRPD analysis and found to be amorphous telithromycin.

| Example | Solvents (volume:volume) |
|---|---|
| 26 | methanol/water (1:1) |
| 27 | acetonitrile/water (1:1) |
| 28 | ethanol/water (1:1) |
| 29 | acetone/water (1:1) |
| 30 | 2-propanol/water (1:1) |
| 31 | tetrahydrofuran/water (1:1) |

While it is apparent that the invention disclosed herein is well calculated to fulfill the objects stated above, it will be appreciated that numerous modifications and embodiments may be devised by those skilled in the art. Therefore, it is intended that the appended claims cover all such modifications and embodiments as falling within the true spirit and scope of the present invention.

We claim:

1. Telithromycin, wherein the telithromycin melts at a range of 175° C. to 185° C.

2. The telithromycin of claim 1, wherein the telithromycin is crystalline.

3. Anhydrous telithromycin, wherein the anhydrous telithromycin melts at a range of 175° C. to 185° C.

4. The anhydrous telithromycin of claim 3, wherein the anhydrous telithromycin is crystalline.

5. Amorphous telithromycin.

6. The amorphous telithromycin of claim 5, having less than about 20% by weight of crystalline telithromycin.

7. The amorphous telithromycin of claim 6, having less than about 10% by weight of crystalline telithromycin.

8. The amorphous telithromycin of claim 7, having less than about 1% by weight of crystalline telithromycin.

9. The amorphous telithromycin of claim 5, characterized by an X-ray powder diffraction pattern substantially as depicted in FIG. 3.

10. The amorphous telithromycin of claim 5, characterized by a differential scanning calorimetry thermogram with peaks at about 174° and about 183° C.

11. A process for preparing the amorphous telithromycin of claim 5, comprising:
a) preparing a mixture of telithromycin in water;
b) precipitating amorphous telithromycin from the mixture; and
c) recovering the precipitated amorphous telithromycin.

12. The process of claim 11, wherein the telithromycin and water of step a) are heated to form a solution, and the obtained solution is then cooled to precipitate the amorphous telithromycin.

13. The process of claim 12, wherein the telithromycin and water of step a) are heated at a temperature of about 70° C. to about reflux.

14. The process of claim 12, wherein the solution is cooled to about room temperature.

15. The process of claim 12, wherein the solution is maintained for a period of about 1 hour to about 5 days prior to recovering the precipitated amorphous telithromycin.

16. A process for preparing the amorphous telithromycin of claim 5, comprising:
a) heating a mixture of telithromycin and methyl tert butyl ether to form a solution;
b) cooling the solution to room temperature to obtain a gel;
c) evaporating the ether to obtain amorphous telithromycin; and
d) recovering the amorphous telithromycin.

17. The process of claim 16, wherein the mixture of telithromycin and ether is heated to reflux to form the solution.

18. A process for preparing the amorphous telithromycin of claim 5, comprising:
a) exposing telithromycin to a solvent to obtain amorphous telithromycin, wherein the solvent is a C₂₋₆ alcohol; and
b) recovering the amorphous telithromycin.

19. The process of claim 18, wherein the C₂₋₆ alcohol is isopropanol.

20. The process of claim 18, wherein the telithromycin is exposed to the solvent for about 1 day to about 40 days.

21. The process of claim 20, wherein the telithromycin is exposed to the solvent for about 40 days.

22. The process of claim 18, wherein the telithromycin and solvent are at a temperature of about 15° C. to about 35° C.

23. A crystalline telithromycin, characterized by an X-ray powder diffraction pattern having peaks at 12.0°, 12.7°, 15.8°, 17.0°, and 19.6° 2η±0.2° 2θ.

24. The crystalline telithromycin of claim 23, wherein the crystalline telithromycin is anhydrous.

25. The crystalline telithromycin of claim 23, further characterized by an X-ray powder diffraction pattern having peaks at 8.2°, 10.4°, 18.3°, 20.7°, and 21.9° 2θ±0.2°2θ.

26. The crystalline telithromycin of claim 25, further characterized by an X-ray powder diffraction pattern substantially as depicted in FIG. 4.

27. The crystalline telithromycin of claim 23, characterized by a differential scanning calorimetry thermogram with peaks at about 155° and about 182° C.

28. A process for preparing the crystalline telithromycin of claim 23, comprising:
a) providing a suspension of amorphous telithromycin in hexane to obtain crystalline telithromycin; and
b) recovering the crystalline telithromycin from the suspension.

29. The process of claim 28, wherein the suspension is maintained for about 15 to about 48 hours to obtain crystalline telithromycin.

30. The process of claim 28, wherein the suspension is maintained at room temperature.

31. A process for preparing the crystalline telithromycin of claim 23, comprising:
a) providing a mixture of amorphous telithromycin and a solvent, wherein the solvent is hexane or a mixture of hexane and water;
b) heating the mixture at a temperature of about 50° C. to about 120° C.;
c) removing the solvent to obtain the crystalline telithromycin; and
d) recovering the crystalline telithromycin.

32. The process of claim 31, wherein the mixture is maintained for at least about half an hour before removing the solvent.

33. The process of claim 31, wherein the solvent is removed by evaporation.

34. The process of claim 33, wherein the solvent is evaporated while heating at a temperature of about 30° C. to about 80° C.

35. A process for preparing the crystalline telithromycin of claim 23, comprising:
a) providing a solution of telithromycin in a cyclic, branched, or unbranched C₄-C₁₀ ether;
b) combining the solution with hexane to obtain a precipitate of the crystalline telithromycin; and
c) recovering the crystalline telithromycin.

36. The process of claim 35, wherein the telithromycin of step a) is amorphous telithromycin.

37. The process of claim 35, wherein the ether is 2-methyl tetrahydrofuran.

38. A crystalline telithromycin, characterized by an X-ray powder diffraction pattern having peaks at 11.9°, 12.1°, 15.8°, 18.0°, and 23.8° 2θ±0.2° 2θ.

39. The crystalline telithromycin of claim 38, wherein the crystalline telithromycin is anhydrous.

40. The crystalline telithromycin of claim 38, further characterized by an X-ray powder diffraction pattern having peaks at 7.8°, 10.1°, 12.9°, 16.4°, 17.6°, 20.5°, 21.3°, and 21.9° 2θ±0.2° 2θ.

41. The crystalline telithromycin of claim 40, further characterized by an X-ray powder diffraction pattern substantially as depicted in FIG. 2.

42. The crystalline telithromycin of claim 38, characterized by a differential scanning calorimetry thermogram with a peak at about 183° C.

43. A process for preparing the crystalline telithromycin of claim 38 comprising:
a) providing a suspension of amorphous telithromycin in a solvent, wherein the solvent is heptane or an aliphatic, branched, or unbranched C₄-C₁₀ ether; and
b) recovering the crystalline telithromycin from the suspension.

44. The process of claim 43, wherein the solvent is heptane, diisopropyl ether, or diethylether.

45. The process of claim 43, wherein the amorphous telithromycin and solvent are heated to form the suspension.

46. The process of claim 45, wherein the amorphous telithromycin and solvent are heated at a temperature of about 40° C. to about reflux.

47. The process of claim 43, wherein the suspension is maintained for at least about 1 hour before recovering the crystalline telithromycin.

48. The process of claim 47, wherein the suspension is maintained for about 1 hour to about 5 hours.

49. A process for preparing the crystalline telithromycin of claim 38 comprising:
a) providing a suspension of amorphous telithromycin in a solvent system of heptane/water, heptane/isopropanol or hexane/isopropanol; and
b) recovering the crystalline telithromycin from the suspension.

50. The process of claim 49, wherein the ratio of solvents in the solvent system is between about 40:1 and about 500:1 volume:volume.

51. The process of claim 49, wherein the amorphous telithromycin and solvent system are heated to form the suspension.

52. The process of claim 49, wherein the amorphous telithromycin and solvent system are heated at a temperature of about 40° C. to about 120° C.

53. The process of claim 49, wherein the suspension is maintained for a period of at least about 4.5 hours to obtain crystalline telithromycin.

54. A process for preparing the crystalline telithromycin of claim 38 comprising:
a) preparing a mixture of amorphous telithromycin in a C₄-C₁₀ aliphatic, branched or unbranched ether, provided that the ether is not methyl tert butyl ether;
b) removing the ether to obtain a precipitate; and
c) recovering crystalline telithromycin from the precipitate.

55. The process of claim 54, wherein the ether is removed by evaporation.

56. The process of claim 54, wherein the amorphous telithromycin and the ether of step a) are heated to form themixture.

57. The process of claim 56, wherein the amorphous telithromycin and the ether are heated at a temperature of about 40° C. to about 120° C.

58. The process of claim 55, wherein the solution is maintained for a period of at least about 4.5 hours before evaporating the solvent.

59. The process of claim 55, wherein the ether is evaporated while heating.

60. The process of claim 59, wherein the ether is evaporated while heating at a temperature of about 30° C. to about 80° C.

61. A process for preparing the crystalline telithromycin of claim 38, comprising:
a) providing a solution of telithromycin in a solvent, wherein the solvent is a cyclic, branched, or unbranched C₄-C₁₀ ether or C₆-C₈ aromatic hydrocarbon;
b) combining the solution with hexane to obtain the crystalline telithromycin; and
c) recovering the crystalline telithromycin.

62. The process of claim 61, wherein the telithromycin of step a) is amorphous telithromycin.

63. The process of claim 61, wherein the solvent is 2-methyl tetrahydrofuran or toluene.

64. A process for preparing the crystalline telithromycin of claim 38, comprising:
a) providing a solution of telithromycin in an aliphatic, branched, or unbranched C₄-C₁₀ ether; and
b) precipitating the crystalline telithromycin from the solution.

65. The process of claim 64, wherein the telithromycin and ether are heated to form the solution.

66. The process of claim 65, wherein the telithromycin and ether are heated at a temperature of about reflux.

67. The process of claim 65, wherein the precipitation is induced by cooling the solution at a temperature of about 0° C.

68. The process of claim 67, wherein the solution is maintained at a temperature of about 0° C. for about 5 hours.

69. The process of claim 64, wherein the telithromycin of step a) is amorphous telithromycin.

70. The process of claim 64, wherein the ether is diethyl ether.

71. A process for preparing the crystalline telithromycin of claim 38, comprising:
a) exposing telithromycin to a solvent to obtain the crystalline telithromycin, wherein the solvent is a C₄₋₁₀ ether; and
b) recovering the crystalline telithromycin.

72. The process of claim 71, wherein the telithromycin of step a) is amorphous telithromycin.

73. The process of claim 71, wherein the solvent is diethyl ether or di-isopropyl ether.

74. The process of claim 71, wherein the telithromycin is exposed to the solvent for about 1 day to about 40 days.

75. The process of claim 71, wherein the telithromycin is exposed to the solvent for about 40 days.

76. The process of claim 71, wherein the telithromycin and solvent are at a temperature of about 15° C. to about 35° C.

77. A process for preparing the crystalline telithromycin of claim 38 comprising heating crystalline telithromycin Form A or amorphous telithromycin.

78. The process of claim 77, wherein the crystalline telithromycin Form A or amorphous telithromycin is heated at a temperature of about 25° C. to about 160° C.

79. The process of claim 77, wherein the crystalline telithromycin Form A is heated in a differential scanning calorimetry furnace with an initial temperature of 30° C. while increasing the temperature at a rate of about 10° C. per minute until a final temperature of 160° C. is reached.

80. The process of claim 77, wherein the amorphous telithromycin is heated in a differential scanning calorimetry furnace with an initial temperature of 25° C. while increasing the temperature at a rate of about 10° C. per minute until a final temperature of 160° C. is reached.

81. A crystalline telithromycin characterized by an X-ray powder diffraction pattern having peaks at 12.0°, 12.7°, 15.8°, 17.0°, and 19.6° 2θ±0.2° 2θ, wherein the crystalline telithromycin has less than about 10% by weight of crystalline telithromycin characterized by an X-ray powder diffraction pattern having peaks at 11.90, 12.1°, 15.8°, 18.0°, and 23.8° 2θ±0.2° 2θ.

82. The crystalline telithromycin of claim 81, characterized by an X-ray powder diffraction pattern free of a peak at about 7.7° 2θ±0.2° 2θ.

83. A process for preparing the crystalline telithromycin of claim 81 comprising:
a) exposing amorphous telithromycin to a solvent to obtain the crystalline telithromycin, wherein the solvent is a C₅-C₈ aliphatic or aromatic hydrocarbon or mixture of C₅-C₈ aliphatic or aromatic hydrocarbon and water; and
b) recovering the crystalline telithromycin.

84. The process of claim 83, wherein the hydrocarbon is hexane, heptane, or a mixture of hexane or heptane with water.

85. The process of claim 83, wherein the amorphous telithromycin is exposed to the solvent for about 1 day to about 40 days.

86. The process of claim 85, wherein the amorphous telithromycin is exposed to the solvent for about 40 days.

87. The process of claim 83, wherein the amorphous telithromycin is exposed to the solvent at a temperature of about 15° C. to about 35° C.
